(19) United States
(12) Patent Application Publication
(10) Pub. No.: US 2004/0143889 A1
(43) Pub. Date: Jul. 29, 2004

(54) Integral swimming/diving goggles

(76) Inventor: Terry Chou, Tainan City (TW)

Correspondence Address: Charles E. Baxley, Esquire, 5th Floor, 59 John Street, New York, NY, 10038, US

(21) Appl. No.: 10/354,817

(22) Filed: Jan. 29, 2003

(51) Int. Cl. A61F 9/02

(52) U.S. Cl. 2/445

(57) ABSTRACT

A pair of swimming/diving goggles includes lenses, two frames, two padding members, a bridge, and a head strap. At least two of the elements are integrally formed as a single piece. Each of the integrally formed elements is made of a basis material and a filling material that are mixed at a desired proportion. The basis materials and the filling materials for each of the integrally formed elements are identical, and the mixing proportions for the integrally formed elements are different from one another, thereby providing the integrally formed elements with respective appropriate rigidity and pull-resistance.

### BACKGROUND OF THE INVENTION

1. Field of the Invention

The present invention relates to a pair of swimming/diving goggles in which at least two of the elements of the pair of swimming/diving goggles are integrally formed to provide improved wearing comfort as well as an aesthetic appearance.

2. Description of the Related Art

FIG. 8 of the drawings illustrates a pair of conventional swimming/diving goggles that includes two rigid, transparent lenses 1′ made of acrylics, glass, etc, two padding members 2′ made of soft material, a bridge 3′, and a head strap 4′. Each padding member 2′ includes a receiving portion 21′ on a side thereof for receiving an associated lens 1′ by stretching the receiving portion 21′, inserting the lens 1′ into the receiving portion 21′, and releasing the stretching force. Formed on the other side of each padding member 2′ is a flange 22′ for intimate contact with an eye socket of a user. The bridge 3′ is made of a material that is harder than the padding members 2′ but softer than the rigid lenses 1′. The bridge 3 is flexible such that the padding members 2′ may be in contact with the eye sockets of the user regardless of the contour of the user. The lenses 1′, the padding members 2′, and the bridge 3′ are made of different materials, which results in an increase in manufacture and management. Further, the pair of swimming/diving goggles could not provide an integral impression, as the lenses 11, the padding members 2′, and the bridge 3′ are separate.

FIGS. 9 and 10 illustrate another pair of conventional swimming/diving googles that includes two lenses 5′, two frames 6′, a bridge 7′, and a head strap 8′. Each lens 1′ is rigid and transparent and made of acrylics, glass, etc. The frames 6′ and the bridge 7′ are integrally formed of a soft material, with the bridge 7′ having a relatively large thickness to provide improved pull-resistance. A padding portion 61′ is formed on an inner side of each frame 6′. However, the padding portions 61′ must be made of soft material for assuring wearing comfort; but the frames 6′ and the bridge 7′ are apt to deform and thus cannot reliably support the lenses 5′ if the frames 6′ and the bridge 7′ are made of the same soft material for the padding portion 61′. On the other hand, if the frames 6′ and the bridge 7′ are made of harder material to improve the pull-resistance and the supporting capability, the padding members 61′ that is integrally formed with the frames 6′ are too hard to provide wearing comfort. As illustrated in FIG. 10, in order to increase the pull-resistance of the soft frames 6′ and the soft bridge 7′, the thickness “b” of each frame 6′ and the thickness “a” of the bridge 7′ are far greater than the thickness “c” of the padding portion 61, which fails to provide an aesthetically pleasing appearance for the pair of swimming/diving goggles. The increased thicknesses of the frames 6′ and the bridge 7′ increase the overall weight of the pair of swimming/diving goggles and cause uncomfortable wearing.

FIGS. 11 and 12 illustrate a further pair of conventional swimming/diving goggles including two lenses 91′, two padding members 92′, a bridge 93′, and a head strap 4. The lenses 91′, the padding members 92′, and the bridge 93′ are integrally made of transparent, slightly soft, and flexible material. Each lens 91′ has a thickness in a front end and two lateral sides thereof to thereby provide a resistance to pull. Further, the annular padding member 92′ is integrally formed on an inner side of the associated lens 91′ and has a thickness smaller than the thickness in the front end and two lateral sides of the lens 91′ to thereby provide required softness and resiliency. Although the thickness in the front end and two lateral sides of the lens 91′ provides required softness and resiliency for the padding members 92′ and required pull-resistance for the lenses 91′ and the bridge 93, the pair of swimming/diving goggles is not good-looking while resulting in an increase in the overall weight of the pair of swimming/diving goggles.

### SUMMARY OF THE INVENTION

An object of the present invention is to provide a pair of swimming/diving goggles in which at least two of the elements of the pair of swimming/diving goggles are integrally formed as a single piece to provide improved wearing comfort as well as an aesthetic appearance. Further, each of the integrally formed elements is made of a basis material and a filling material that are mixed at a desired proportion. The basis materials and filling materials for each of the integrally formed elements are identical, but the mixing proportions for the respective integrally formed elements are different from one another. Thus, the integrally formed elements possess respective appropriate rigidity and pull-resistance.

Other objects, advantages, and novel features of the invention will become more apparent from the following detailed description when taken in conjunction with the accompanying drawings.

### BRIEF DESCRIPTION OF THE DRAWINGS

FIG. 1 is a top view, partly sectioned, of a first embodiment of a pair of swimming/diving goggles in accordance with the present invention.

FIG. 2 is a top view, partly sectioned, of a second embodiment of a pair of swimming/diving goggles in accordance with the present invention.

FIG. 3 is a top view, partly sectioned, of a third embodiment of a pair of swimming/diving goggles in accordance with the present invention.

FIG. 4 is a top view, partly sectioned, of a fourth embodiment of a pair of swimming/diving goggles in accordance with the present invention.

FIG. 5 is a top view, partly sectioned, of a fifth embodiment of a pair of swimming/diving goggles in accordance with the present invention.

FIG. 6 is a top view, partly sectioned, of a sixth embodiment of a pair of swimming/diving goggles in accordance with the present invention.

FIG. 7 is a top view, partly sectioned, of a seventh embodiment of a pair of swimming/diving goggles in accordance with the present invention.

FIG. 8 is a perspective view, partly exploded, of a pair of conventional swimming/diving goggles.

FIG. 9 is a perspective view of another pair of conventional swimming/diving goggles.

FIG. 10 is a top view, partly sectioned, of the pair of conventional swimming/diving goggles in FIG. 9.

FIG. 11 is an exploded perspective view of a further pair of conventional swimming/diving goggles.

FIG. 12 is a top view, partly sectioned, of the pair of conventional swimming/diving goggles in FIG. 11.

### DETAILED DESCRIPTION OF THE PREFERRED EMBODIMENTS

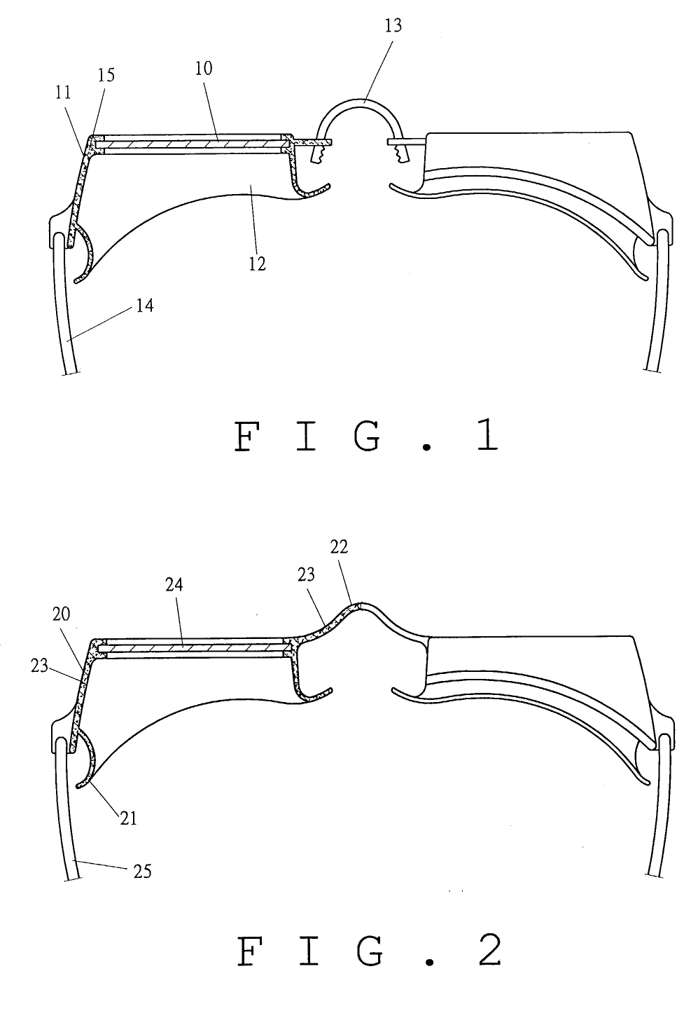

FIG. 1 illustrates a first embodiment of a pair of swimming/diving goggles in accordance with the present invention. In this embodiment, the pair of swimming/diving goggles includes two lenses 10, two frames 11, two padding members 12, a bridge 13, and a head strap 14.

The frames 11 and the padding members 12 are integrally formed as a single piece. Each of the frames 11 and the padding members 12 is made of a basis material and a filling material 15 that are mixed at a desired proportion. The basis material is selected from a group consisting of silicon rubber and rubber. The filling material 15 is preferably sintered silica. The basis materials and the filling materials for each of the integrally formed elements are identical, and the mixing proportions for the integrally formed elements are different from one another. Thus, frames 11 and the padding members 12 that are integrally formed possess respective appropriate rigidity and pull-resistance. For example, using the frame 11 as a reference for rigidity, the rigidity of the padding member 12 is 66% of that of the frame 11.

In manufacture, the raw material for the frames 11 and the raw material for the padding members 12 are poured into a mold (not shown) to allow integral formation of the frames 11 and the padding members 12 after the raw materials harden. The padding members 12 possess desired softness, and the frames 11 possess sufficient resistance to pull without largely increasing the thickness thereof. The pair of swimming/diving goggles thus formed is light in weight and provides an aesthetically pleasing appearance in an overall view.

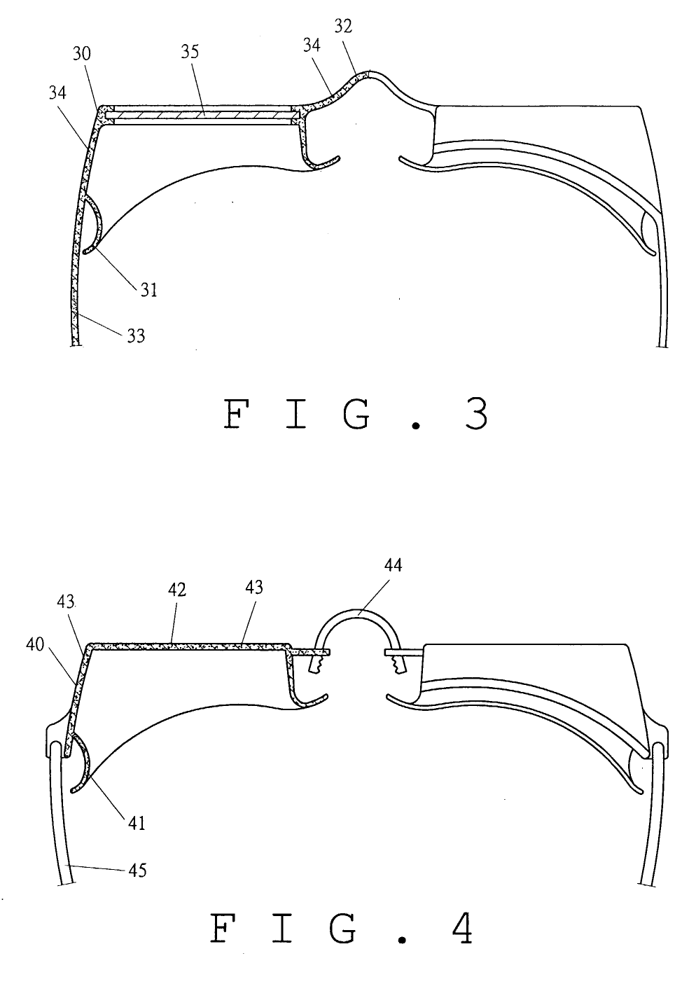

FIG. 2 illustrates a second embodiment of a pair of swimming/diving goggles in accordance with the present invention. In this embodiment, the pair of swimming/diving goggles includes two lenses 24, two frames 20, two padding members 21, a bridge 22, and a head strap 25.

The frames 20, the padding members 21, and the bridge 22 are integrally formed as a single piece. Each of the frames 20, the padding members 21, and the bridge 22 is made of a basis material and a filling material 23 that are mixed at a desired proportion. The basis material is selected from a group consisting of silicon rubber and rubber. The filling material 23 is preferably sintered silica. The basis materials and the filling materials for each of the integrally formed elements are identical, and the mixing proportions for the integrally formed elements are different from one another. Thus, frames 20, the padding members 21, and the bridge 22 that are integrally formed possess respective appropriate rigidity and pull-resistance. For example, using the frame 20 as a reference for rigidity, the rigidity of the padding member 21 is 66% of that of the frame 20, and the rigidity of the bridge 22 is 125% of that of the frame 20.

In manufacture, the raw materials respectively for the frames 20, the padding members 21, and the bridge 22 are poured into a mold (not shown) to allow integral formation of frames 20, the padding members 21, and the bridge 22 after the raw materials harden. The padding members 21 possess desired softness, and the frames 20 and the bridge 22 possess sufficient resistance to pull and desired rigidity. The pair of swimming/diving goggles thus formed is light in weight and provides an aesthetically pleasing appearance in an overall view.

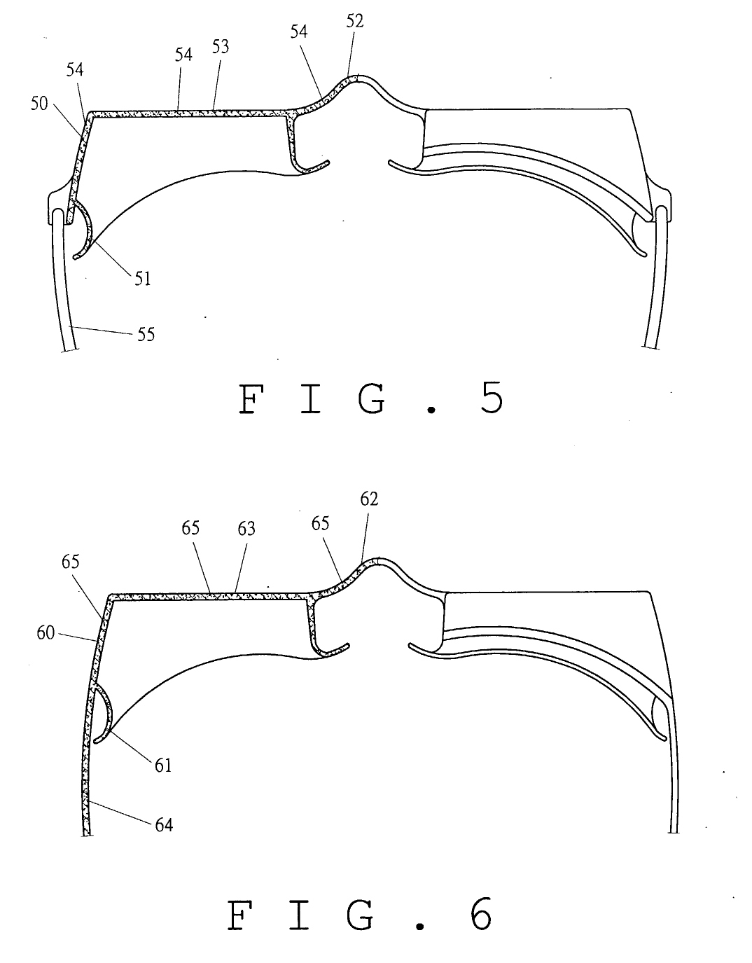

FIG. 3 illustrates a third embodiment of a pair of swimming/diving goggles in accordance with the present invention. In this embodiment, the pair of swimming/diving goggles includes two lenses 35, two frames 30, two padding members 31, a bridge 32, and a head strap 33.

The frames 30, the padding members 31, the bridge 32, and the head strap 33 are integrally formed as a single piece. Each of the frames 30, the padding members 31, the bridge 32, and the head strap 33 is made of a basis material and a filling material 34 that are mixed at a desired proportion. The basis material is selected from a group consisting of silicon rubber and rubber. The filling material 34 is preferably sintered silica. The basis materials and the filling materials for each of the integrally formed elements are identical, and the mixing proportions for the integrally formed elements are different from one another. Thus, the frames 30, the padding members 31, the bridge 32, and the head strap 33 that are integrally formed possess respective appropriate rigidity and pull-resistance. For example, using the frame 30 as a reference for rigidity, the rigidity of the padding member 31 is 66% of that of the frame 30, the rigidity of the bridge 32 is 125% of that of the frame 30, and the rigidity of the head strap 33 is 50% of that of the frame 30.

In manufacture, the raw materials respectively for the frames 30, the padding members 31, the bridge 32, and the head strap 33 are poured into a mold (not shown) to allow integral formation of frames 30, the padding members 31, the bridge 32, and the head strap 33 after the raw materials harden. The padding members 31 and the head strap 33 possess desired softness, and the frames 30 and the bridge 32 possess desired pull-resistance and desired rigidity. The pair of swimming/diving goggles thus formed is light in weight and provides an aesthetically pleasing appearance in an overall view.

FIG. 4 illustrates is a fourth embodiment of a pair of swimming/diving goggles in accordance with the present invention. In this embodiment, the pair of swimming/diving goggles includes two lenses 42, two frames 40, two padding members 41, a bridge 44, and a head strap 45.

The frames 40, the padding members 41, and the lenses 42 are integrally formed as a single piece. Each of the frames 40, the padding members 41, and the lenses 42 is made of a basis material and a filling material 43 that are mixed at a desired proportion. The basis material is selected from a group consisting of silicon rubber and rubber. The filling material 43 is preferably sintered silica. The basis materials and the filling materials for each of the integrally formed elements are identical, and the mixing proportions for the integrally formed elements are different from one another. Thus, the frames 40, the padding members 41, and the lenses 42 that are integrally formed possess respective appropriate rigidity and pull-resistance. For example, using the frame 40 as a reference for rigidity, the rigidity of the padding member 41 is 66% of that of the frame 30, and the rigidity of the lenses 42 is 150% of that of the frame 40.

In manufacture, the raw materials respectively for the frames 40, the padding members 41, and the lenses 42 are poured into a mold (not shown) to allow integral formation of frames 40, the padding members 41, and the lenses 42 after the raw materials harden. The padding members 41 possess desired softness, the frames 40 possess desired pull-resistance, and the lenses 42 posses desired rigidity and desired pull-resistance. The pair of swimming/diving goggles thus formed is light in weight and provides an aesthetically pleasing appearance in an overall view.

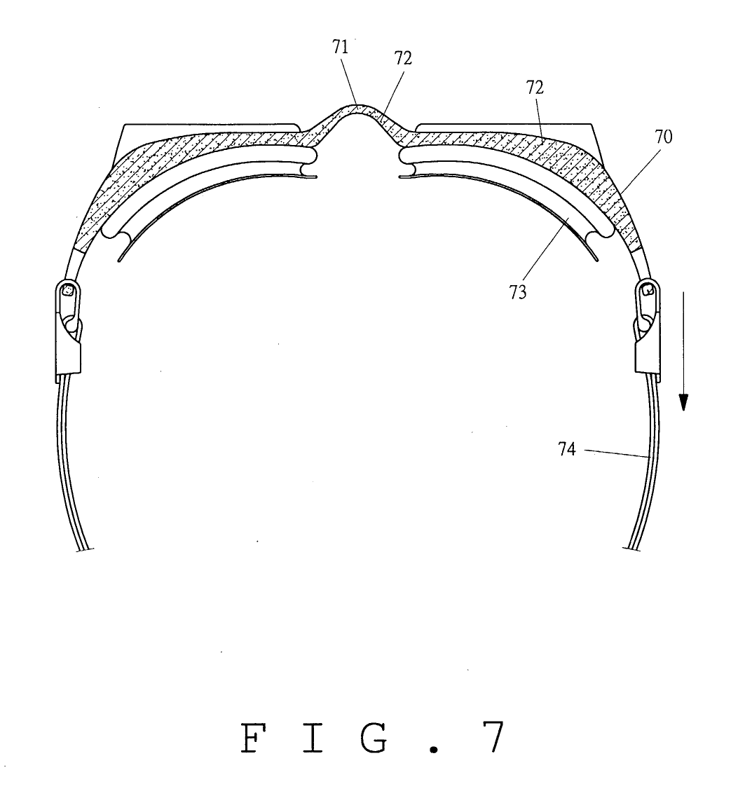

FIG. 5 illustrates a fifth embodiment of a pair of swimming/diving goggles in accordance with the present invention. In this embodiment, the pair of swimming/diving goggles includes two lenses 53, two frames 50, two padding members 51, a bridge 52, and a head strap 55.

The frames 50, the padding members 51, the bridge 52, and the lenses 53 are integrally formed as a single piece. Each of the frames 50, the padding members 51, the bridge 52, and the lenses 53 is made of a basis material and a filling material 54 that are mixed at a desired proportion. The basis material is selected from a group consisting of silicon rubber and rubber. The filling material 54 is preferably sintered silica. The basis materials and the filling materials for each of the integrally formed elements are identical, and the mixing proportions for the integrally formed elements are different from one another. Thus, the frames 50, the padding members 51, the bridge 52, and the lenses 53 that are integrally formed possess respective appropriate rigidity and pull-resistance. For example, using the frame 50 as a reference for rigidity, the rigidity of the padding member 51 is 66% of that of the frame 50, the rigidity of the bridge 52 is 125% of that of the frame 50, and the rigidity of the lens 53 is 50% of that of the frame 50.

In manufacture, the raw materials respectively for the frames 50, the padding members 51, the bridge 52, and the lenses 53 are poured into a mold (not shown) to allow integral formation of frames 50, the padding members 51, the bridge 52, and the lenses 53 after the raw materials harden. The padding members 51 possess desired softness, the frames 50 and the bridge 52 possess desired pull-resistance, and the lenses 53 possess desired rigidity and desired pull-resistance. The pair of swimming/diving goggles thus formed is light in weight and provides an aesthetically pleasing appearance in an overall view.

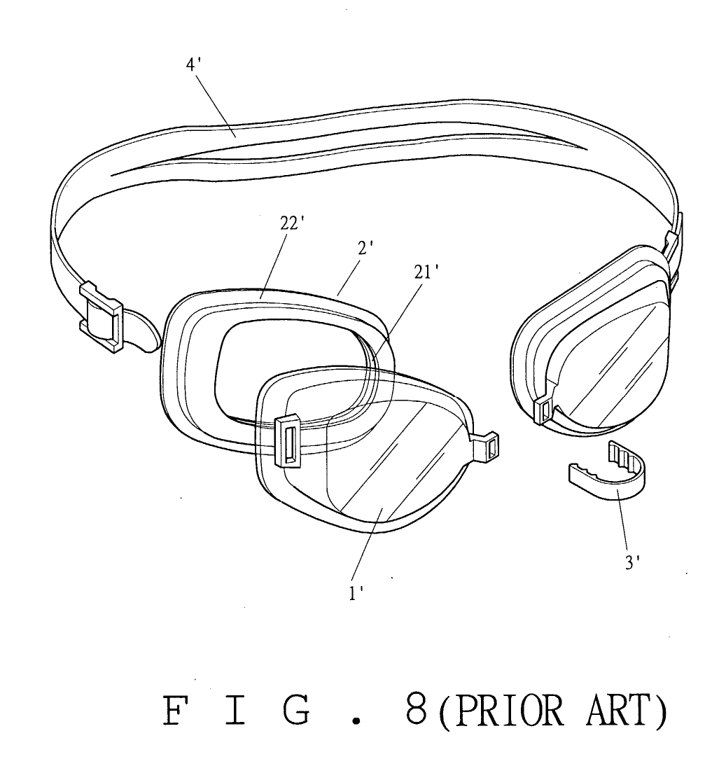

FIG. 6 illustrates a sixth embodiment of a pair of swimming/diving goggles in accordance with the present invention. In this embodiment, the pair of swimming/diving goggles includes two lenses 63, two frames 60, two padding members 61, a bridge 62, and a head strap 64.

The frames 60, the padding members 61, the bridge 62, the lenses 63, and the head strap 64 are integrally formed as a single piece. Each of the frames 60, the padding members 61, the bridge 62, the lenses 63, and the head strap 64 is made of a basis material and a filling material 65 that are mixed at a desired proportion. The basis material is selected from a group consisting of silicon rubber and rubber. The filling material 65 is preferably sintered silica. The basis materials and the filling materials for each of the integrally formed elements are identical, and the mixing proportions for the integrally formed elements are different from one another. Thus, the frames 60, the padding members 61, the bridge 62, the lenses 63, and the head strap 64 that are integrally formed possess respective appropriate rigidity and pull-resistance. For example, using the frame 60 as a reference for rigidity, the rigidity of the padding member 61 is 66% of that of the frame 60, the rigidity of the bridge 62 is 125% of that of the frame 60, the rigidity of the lens 63 is 150% of that of the frame 60, and the rigidity of the head strap 64 is 50% of that of the frame 60.

In manufacture, the raw materials respectively for the frames 60, the padding members 61, the bridge 62, the lenses 63, and the head strap 64 are poured into a mold (not shown) to allow integral formation of frames 60, the padding members 61, the bridge 62, the lenses 63, and the head strap 64 after the raw materials harden. The padding members 61 and the head strap 64 possess desired softness, the frames 60 and the bridge 62 possess desired pull-resistance, and the lenses 63 possess desired rigidity. The pair of swimming/diving goggles thus formed is light in weight and provides an aesthetically pleasing appearance in an overall view.

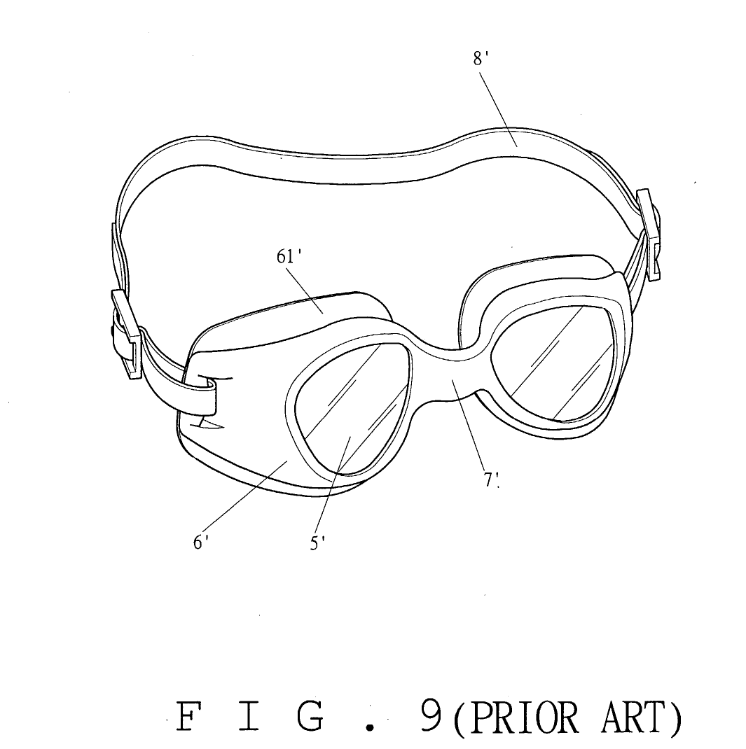

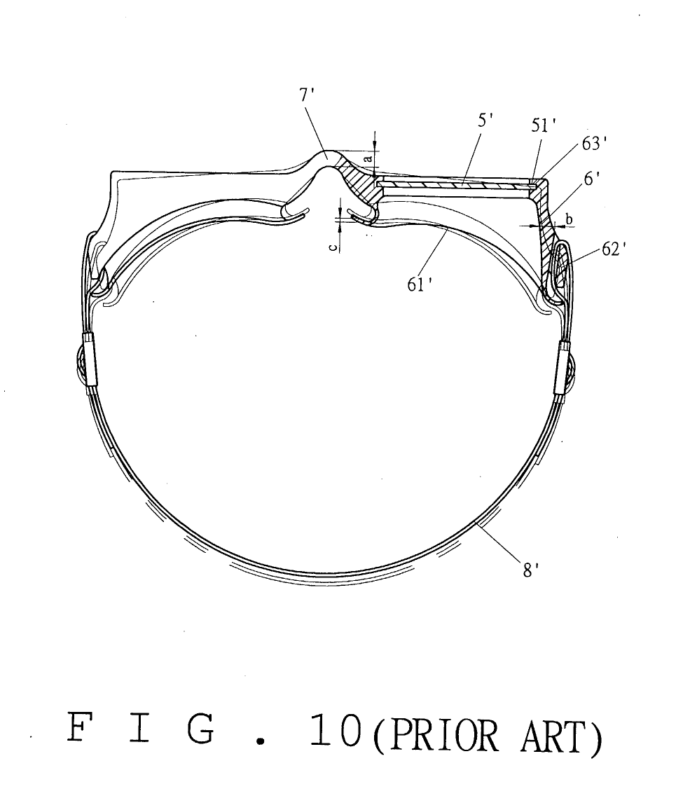

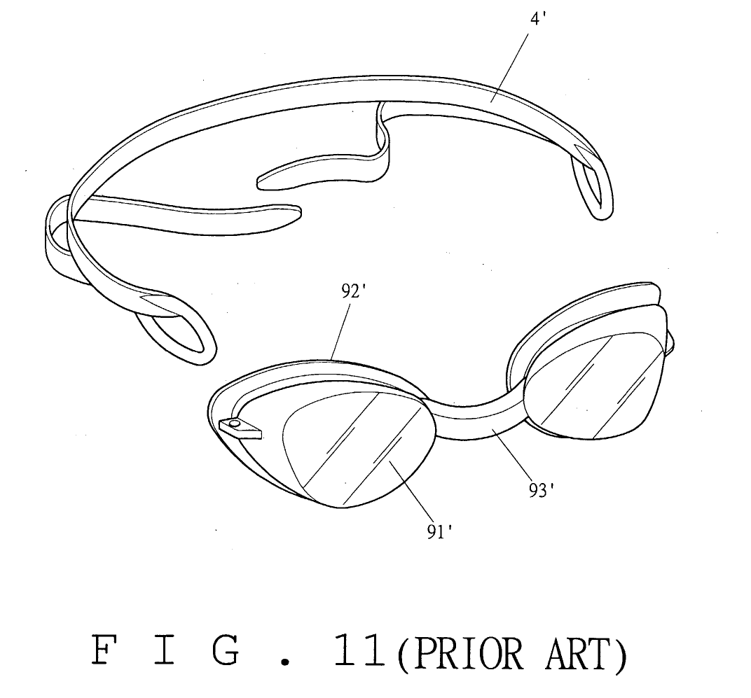

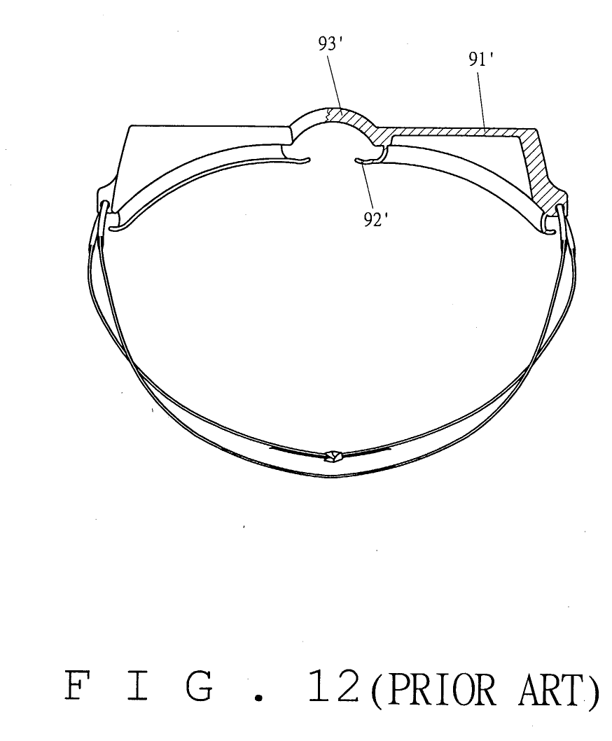

FIG. 7 illustrates a seventh embodiment of a pair of swimming/diving goggles in accordance with the present invention. In this embodiment, the pair of swimming/diving goggles includes two lenses (not shown), two frames 70, two padding members 73, a bridge 71, and a head strap 74.

The frames 70 and the bridge 71 are integrally formed as a single piece. Each of the frames 70 and the bridge 71 is made of a basis material and a filling material 72 that are mixed at a desired proportion. The basis material is selected from a group consisting of silicon rubber and rubber. The filling material 72 is preferably sintered silica. The basis materials and the filling materials for each of the integrally formed elements are identical, and the mixing proportions for the integrally formed elements are different from one another. Thus, frames 70 and the bridge 71 that are integrally formed possess respective appropriate rigidity and pull-resistance. For example, using the frame 70 as a reference for rigidity, the rigidity of the bridge 71 is 125% of that of the frame 70.

In manufacture, the raw materials respectively for the frames 70 and the bridge 71 are poured into a mold (not shown) to allow integral formation of frames 70 and the bridge 71 after the raw materials harden. The frames 70 and the bridge 71 possess desired rigidity and desired pull-resistance. The pair of swimming/diving goggles thus formed is light in weight and provides an aesthetically pleasing appearance in an overall view.

According to the above description, it is appreciated that the integrally formed elements of the pair of swimming/diving goggles in accordance with the present invention possess desired rigidity and desired pull-resistance without largely increasing the thicknesses thereof. The pair of swimming/diving goggles thus formed is light in weight and provides an aesthetically pleasing appearance in an overall view, as the thicknesses of the integrally formed elements have little difference.

Although the invention has been explained in relation to its preferred embodiment, it is to be understood that many other possible modifications and variations can be made without departing from the scope of the invention as hereinafter claimed.

What is claimed is:

1. A pair of swimming/diving goggles comprising a plurality of elements including two lenses, two frames, two padding members, a bridge, and a head strap, at least two of the elements being integrally formed as a single piece, each of the integrally formed elements being made of a basis material and a filling material that are mixed at a desired proportion, the basis materials and the filling materials for each of the integrally formed elements being identical, the mixing proportions for the integrally formed elements being different from one another, thereby providing the integrally formed elements with respective appropriate rigidity and pull-resistance.

2. The pair of swimming/diving goggles as claimed in claim 1, wherein the frames and the padding members are integrally formed as a single piece, and wherein a rigidity of the padding member is 66% of that of the frame.

3. The pair of swimming/diving goggles as claimed in claim 1, wherein the frames, the padding members, and the bridge are integrally formed as a single piece, and wherein a rigidity of the padding member is 66% of that of the frame, and a rigidity of the bridge is 125% of that of the frame.

4. The pair of swimming/diving goggles as claimed in claim 1, wherein the frames, the padding members, the bridge, and the head strap are integrally formed as a single piece, and wherein a rigidity of the padding member is 66% of that of the frame, a rigidity of the bridge is 125% of that of the frame, and a rigidity of the head strap is 50% of that of the frame.

5. The pair of swimming/diving goggles as claimed in claim 1, wherein the frames, the padding members, and the lenses are integrally formed as a single piece, and wherein a rigidity of the padding member is 66% of that of the frame, and a rigidity of the lenses is 150% of that of the frame.

6. The pair of swimming/diving goggles as claimed in claim 1, wherein the frames, the padding members, the bridge, and the lenses are integrally formed as a single piece, and wherein a rigidity of the padding member is 66% of that of the frame, a rigidity of the bridge is 125% of that of the frame, and a rigidity of the lens is 50% of that of the frame.

7. The pair of swimming/diving goggles as claimed in claim 1, wherein the frames, the padding members, the bridge, the lenses, and the head strap are integrally formed as a single piece, and wherein a rigidity of the padding member is 66% of that of the frame, a rigidity of the bridge is 125% of that of the frame, a rigidity of the lens is 150% of that of the frame, and a rigidity of the head strap is 50% of that of the frame.

8. The pair of swimming/diving goggles as claimed in claim 1, wherein the frames and the bridge are integrally formed as a single piece, and wherein a rigidity of the bridge is 125% of that of the frame.

9. The pair of swimming/diving goggles as claimed in claim 1, wherein the basis material is selected from a group consisting of silicon rubber and rubber.

10. The pair of swimming/diving goggles as claimed in claim 1, wherein the filling material is sintered silica.
